(19) United States
(12) Patent Application Publication
(10) Pub. No.: US 2026/0090798 A1
(43) Pub. Date: Apr. 2, 2026

(54) HANDHELD TISSUE SUTURING DEVICE

(71) Applicant: POSTECH RESEARCH AND BUSINESS DEVELOPMENT FOUNDATION, Pohang-si (KR)

(72) Inventors: Wankyun CHUNG, Pohang-si (KR); Won Bin CHOI, Pohang-si (KR); Hyunggon SHIN, Pohang-si (KR)

(73) Assignee: POSTECH RESEARCH AND BUSINESS DEVELOPMENT FOUNDATION, Pohang-si (KR)

(21) Appl. No.: 19/233,409

(22) Filed: Jun. 10, 2025

(30) Foreign Application Priority Data
Oct. 2, 2024 (KR) 10-2024-0134244

(51) Int. Cl. A61B 17/04

(52) U.S. Cl. CPC A61B 17/0483; A61B 17/0482

(57) ABSTRACT

A handheld tissue suturing device includes a handle portion extending in a first direction, a rotating body including a first body part and a second body part positioned on opposite sides of each other, wherein the first body part is connected to a connection end of the handle portion and is configured to be rotatable around a rotation axis extending in the first direction, a finger part including a pivot end connected to the second body part of the rotating body and configured to be pivotable around a pivot axis extending in a second direction intersecting the rotation axis, a vacuum tweezer fixed to the finger part, and a suction tube extending from the vacuum tweezer through the rotating body to the handle portion and forming an air suction path.

### CROSS-REFERENCE TO RELATED APPLICATIONS

This application is based on Korean Patent Application No. 10-2024-0134244, filed on Oct. 2, 2024, the entire contents of which are incorporated herein by reference.

### BACKGROUND

### 1. Field

This disclosure relates to a handheld tissue suturing device.

### 2. Description of the Related Art

For tissue suturing during medical surgeries, medical forceps and suturing needles are mainly used. These traditional suturing devices involve the user directly inserting the needle into the tissue and passing the suture thread through, and the suturing results may vary greatly depending on the surgeon's skill level. In particular, the suturing method must vary according to the type and shape of the wound, which often relies on the surgeon's experience because of the many technical difficulties in implementing an accurate suture pattern.

Existing suturing devices do not allow observation of the inside of the tissue during the suturing process, so the surgeon must rely on intuition to infer the position of the needle tip when the needle passes through the tissue. This makes it difficult to accurately determine the path of the needle through the tissue, and there are limitations in precisely controlling the depth and length of the suture. Additionally, hand tremors or vibrations that may occur during surgery are directly transmitted to the needle, interfering with accurate needle manipulation and adversely affecting suture quality.

In particular, for corneal suturing in ophthalmic surgery, the accuracy of suturing greatly affects the surgical outcome. Incorrect suture depth or length may cause post-operative complications, and in severe cases, reoperation may be necessary. For example, if the suture depth is too deep, damage to the cornea may occur, and conversely, if the suture length is too short, ocular fluid leakage may occur. Additionally, if the suture pattern is not uniform, it may cause visual problems such as astigmatism after surgery. Therefore, there is a need for new technology that can accurately control the depth and length of sutures when suturing wounds, and the development of innovative suturing devices that can solve these problems is desirable.

### SUMMARY

One aspect of an embodiment is to provide a handheld tissue suturing device that may minimize processes or operations during suturing surgery and facilitate the manipulation of the attached vacuum tweezer.

However, the problems that the embodiments of the present disclosure aim to solve are not limited to the above-mentioned problems and may be variously expanded within the scope of the technical concept included in the present disclosure.

A handheld tissue suturing device according to an embodiment includes a handle portion extending in a first direction, a rotating body including a first body part and a second body part positioned on opposite sides of each other, wherein the first body part is connected to a connection end of the handle portion and is configured to be rotatable around a rotation axis extending in the first direction, a finger part including a pivot end connected to the second body part of the rotating body and configured to be pivotable around a pivot axis extending in a second direction intersecting the rotation axis, a vacuum tweezer fixed to the finger part, and a suction tube extending from the vacuum tweezer through the rotating body to the handle portion and forming an air suction path.

The suction tube may be configured to extend through at least a portion of the rotating body along the rotation axis.

The connection end of the handle portion and the first body part of the rotating body may have a male-female coupling structure capable of relative rotation around the rotation axis.

The connection end of the handle portion may include a female part, and the first body part of the rotating body may include a male part.

The male-female coupling structure may have a rotation limiting structure configured to limit the rotation range according to the rotation direction.

The rotation limiting structure may include a protrusion having a first central angle in the rotation direction, and a recess having a second central angle larger than the first central angle, wherein the protrusion is accommodated in the recess to move in the rotation direction.

The handle portion and the rotating body each may have a cavity inside, and the cavities may be configured to have a coaxial central axis and to communicate with each other.

The cavities may be configured to be connected at adjacent portions of the handle portion and the rotating body.

The central axis of the cavities may extend in the first direction and may be aligned in line with the rotation axis.

The second body part of the rotating body and the pivot end of the finger part may have a position fixing structure that limits the driving range in the pivot direction and fixes the position.

The position fixing structure may include an inner diameter part positioned around the pivot axis and having grooves on its outer circumference, and an outer diameter part arranged to surround a portion of the outer circumference of the inner diameter part and having protrusions corresponding to the grooves.

The outer diameter part may include a pair of arms extending in opposite directions along the outer circumference of the inner diameter part, and the end of each of the pair of arms may include the protrusion protruding toward the inner diameter part.

The pair of arms may have elasticity to move in the radial direction of the inner diameter part.

The vacuum tweezer may include a first piece part and a second piece part, each coupled on opposite sides of the finger part.

The first piece part may include a first coupling plate attached adjacent to one side of the finger part and a first contour forming plate bent and extended from the first coupling plate, and the second piece part may include a second coupling plate attached adjacent to the other side of the finger part and a second contour forming plate bent and extended from the second coupling plate, wherein the first contour forming plate and the second contour forming plate extend to face each other and are arranged so that their respective opposing ends overlap each other.

A needle hole penetrating the first coupling plate and the second coupling plate is located at the longitudinal lower end of each of the first coupling plate and the second coupling plate, and the needle hole has a first opening section and a second opening section connected to the first opening section and open from the lower edge of the first coupling plate or the second coupling plate, and the width of the connecting portion of the first opening section and the second opening section may be smaller than the maximum width of the first opening section.

The first contour forming plate and the second contour forming plate have a first concave part and a second concave part with asymmetric contour shapes adjacent to the lower end of edges facing each other, and the first concave part and the second concave part may be integrated to form a suturing area when the first piece part and the second piece part are coupled.

The handle portion may have at least a pair of tube receiving grooves that accommodate the suction tube and are blocked from each other inside, and the handle portion may include a tube switch where a portion of the suction tube connecting between the at least pair of tube receiving grooves is arranged to pass along the outer surface of the handle portion.

The suction tube may include a first suction tube and a second suction tube forming different suction paths, and the handle portion may include a multiple tube control switch configured to individually open or close the first suction tube and the second suction tube.

The multiple tube control switch may include a tube fixing frame supporting the first suction tube and the second suction tube, and a pressing member arranged to face the tube fixing frame with the first suction tube and the second suction tube interposed.

According to the handheld tissue suturing device of the embodiment, processes or operations during suturing surgery can be minimized, and the manipulation of the mounted vacuum tweezer can be made simple and easy.

### BRIEF DESCRIPTION OF THE DRAWINGS

FIG. 1 is a perspective view illustrating a handheld tissue suturing device according to an embodiment.

FIG. 2 is a partial exploded perspective view illustrating the male-female coupling structure of the handle portion and the rotating body of the handheld tissue suturing device shown in FIG. 1.

FIG. 3 is a partial exploded perspective view illustrating the protrusion and recess for limiting the rotation range of the male-female coupling structure shown in FIG. 2.

FIG. 4 is a view illustrating the operation of the handheld tissue suturing device shown in FIG. 1.

FIG. 5 is a partial side view illustrating the position fixing structure of the rotating body and the finger part of the handheld tissue suturing device shown in FIG. 1.

FIG. 6 is a partial exploded perspective view illustrating the finger part and the vacuum tweezer of the handheld tissue suturing device shown in FIG. 1.

FIG. 7 is a view illustrating the configuration of the vacuum tweezer of the handheld tissue suturing device shown in FIG. 1.

FIG. 8 is an image photograph showing the state of holding the handheld tissue suturing device shown in FIG. 1 by hand.

FIG. 9 is a perspective view illustrating a handheld tissue suturing device according to another embodiment.

FIG. 10 is a partial exploded perspective view illustrating the finger part and the vacuum tweezer of the handheld tissue suturing device shown in FIG. 9.

FIG. 11(a) to 11(c) provide views illustrating the configuration of the vacuum tweezer of the handheld tissue suturing device shown in FIG. 9.

FIG. 12 is a perspective view illustrating the multiple tube control switch of the handheld tissue suturing device shown in FIG. 9.

FIG. 13 is a schematic view illustrating the operation of the multiple tube control switch shown in FIG. 12.

FIG. 14 is a perspective view illustrating a modification of the multiple tube control switch shown in FIG. 12.

### DETAILED DESCRIPTION

Hereinafter, embodiments of the present disclosure will be described in detail so that those skilled in the art to which the present disclosure pertains may easily carry out the present disclosure with reference to the accompanying drawings. In the drawings, parts irrelevant to the description have been omitted for clarity of explanation of the present disclosure, and the same or similar components are denoted by the same reference numerals throughout the specification. Also, in the accompanying drawings, some components may be exaggerated, omitted, or schematically illustrated, and the size of each component does not entirely reflect its actual size.

The accompanying drawings are only for easy understanding of the embodiments disclosed in this specification, and the technical concept disclosed in this specification is not limited by the accompanying drawings, and it should be understood to include all modifications, equivalents or substitutes included in the spirit and technical scope of the present disclosure.

Terms including ordinal numbers such as first, second, and the like may be used to describe various components, but the components are not limited by these terms. These terms are used only for the purpose of distinguishing one component from another component.

Also, when a part such as a layer, film, region, plate, or the like is said to be “on” or “above” another part, it includes not only the case where it is “directly on” the other part, but also the case where there is another part in between. Conversely, when a part is said to be “directly on” another part, it means that there are no other parts in between. Also, when a part is said to be “on” or “above” a reference part, it is located above or below the reference part, and does not necessarily mean that it is positioned “on” or “above” in the direction opposite to gravity.

Throughout the specification, terms such as “include” or “have” are intended to specify the presence of features, numbers, steps, operations, components, parts, or combinations thereof described in the specification, rather than excluding in advance the possibility of the presence or addition of one or more other features, numbers, steps, operations, components, parts, or combinations thereof. Therefore, when a part is said to “include” a component, it means that it may include other components as well, rather than excluding other components, unless there is a specific statement to the contrary.

Also, throughout the specification, when it is said “in plan view,” it means when the target part is viewed from above, and when it is said “in cross-sectional view,” it means when a cross section of the target part cut vertically is viewed from the side.

Also, throughout the specification, when it is said to be “connected,” it means not only that two or more components are directly connected, but also that two or more components are indirectly connected through other components, that they are physically connected as well as electrically connected, or that they may be referred to by different names according to position or function but are integral.

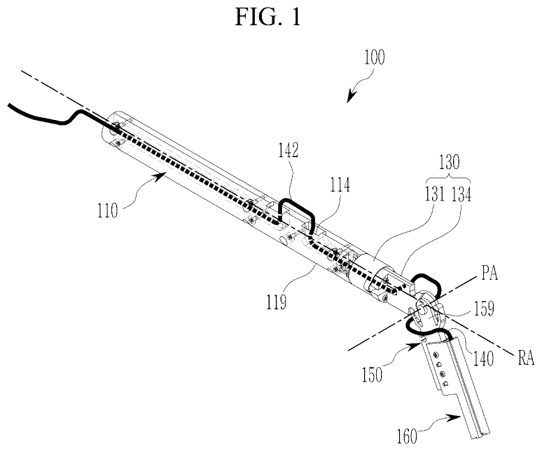

FIG. 1 is a perspective view illustrating a handheld tissue suturing device according to an embodiment.

Referring to FIG. 1, the handheld tissue suturing device 100 according to this embodiment includes a handle portion 110, a rotating body 130, and a finger part 150. A vacuum tweezer 160 used for tissue suturing is fixed to the finger part 150, and a suction tube 140 extending from the vacuum tweezer 160 to the handle portion 110 may be connected to the vacuum tweezer 160.

The handle portion 110 may have a rod structure extending lengthwise in a first direction. For example, the handle portion 110 may be formed as a cylinder having a circular cross-section. The handle portion 110 may have a tube receiving groove 114 to accommodate the suction tube 140.

The rotating body 130 includes a first body part 131 and a second body part 134 positioned on opposite sides of each other, and the first body part 131 may be connected to a connection end 119 of the handle portion 110. Here, the rotating body 130 may be configured to be rotatable around a rotation axis RA extending in the first direction. That is, the connection end 119 of the handle portion 110 and the first body part 131 of the rotating body 130 are connected to each other, and the rotating body 130 has a structure that can relatively rotate with respect to the handle portion 110.

The first body part 131 and the second body part 134 of the rotating body 130 may each be formed by the coupling of separate parts. The first body part 131 may have a through hole 131a penetrated along the rotation axis RA. The suction tube 140 may pass through the through hole 131a. Because the through hole 131a extends along the rotation axis RA and the suction tube 140 passes along this through hole 131a, the suction tube 140 may not become twisted or kinked even if the rotating body 130 rotates around the rotation axis RA.

The finger part 150 includes a pivot end 159 capable of pivoting movement, and may be connected to the second body part 134 of the rotating body 130 through this pivot end 159. The pivot end 159 may be pivot-driven around a pivot axis PA extending in a second direction intersecting the first direction. Therefore, the pivot axis PA and the rotation axis RA may extend in directions that intersect each other.

The vacuum tweezer 160 may be fixed to the opposite side of the pivot end 159 of the finger part 150. Therefore, the vacuum tweezer 160 may be displaced together as the finger part 150 changes position while pivoting.

The suction tube 140 may be configured to be connected to the vacuum tweezer 160 to provide suction power. The suction tube 140 may extend from the vacuum tweezer 160 through the rotating body 130 to the handle portion 110 and form an air suction path. Here, the suction tube 140 may extend through at least a portion of the rotating body 130 along the rotation axis RA. The suction tube 140 may be made of a flexible material.

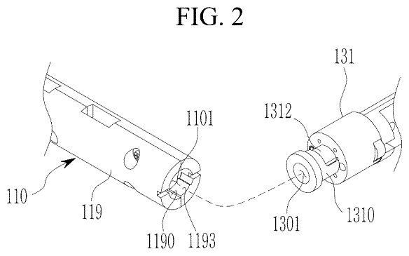

FIG. 2 is a partial exploded perspective view illustrating the male-female coupling structure of the handle portion and the rotating body of the handheld tissue suturing device shown in FIG. 1.

Referring to FIG. 2, the connection end 119 of the handle portion 110 and the first body part 131 of the rotating body 130 may have a male-female coupling structure capable of relative rotation around the rotation axis RA. The connection end 119 of the handle portion 110 includes a female part 1190, and the first body part 131 of the rotating body 130 may include a male part 1310. The rotating body 130 may be coupled to the handle portion 110 by inserting the male part 1310 into the female part 1190.

The male-female coupling structure connecting the handle portion 110 and the rotating body 130 may have a rotation limiting structure configured to limit the rotation range according to the rotation direction. The rotation limiting structure may include a protrusion 1193 having a first central angle in the rotation direction, and a recess 1312 having a second central angle larger than the first central angle. The protrusion 1193 may be accommodated in the recess 1312 to move in the rotation direction.

The protrusion 1193 for limiting the rotation range may be formed on the female part 1190 of the handle portion 110. The protrusion 1193 may protrude from the inner surface of the female part 1190 toward the central axis. The protrusion 1193 may be arranged to protrude from a portion of the inner circumference of the female part 1190. The recess 1312 for limiting the rotation range may be formed on the male part 1310 of the rotating body 130. The recess 1312 may protrude radially outward from the outer surface of the male part 1310. The recess 1312 may be arranged to protrude from a portion of the outer circumference of the male part 1310.

Because the second central angle is greater than the first central angle, the inner circumferential length of the recess 1312 may be formed to be longer than the outer circumferential length of the protrusion 1193. Therefore, the recess 1312 can secure space for the protrusion 1193 to move in the circumferential direction.

The handle portion 110 and the rotating body 130 each may have cavities 1101 and 1301 inside. The cavity 1101 of the handle portion 110 and the cavity 1301 of the rotating body 130 may be configured to have a coaxial central axis and to communicate with each other. Therefore, the cavities 1101 and 1301 may be configured to be connected at adjacent portions of the handle portion 110 and the rotating body 130.

The central axis of the cavities 1101 and 1301 may extend in the first direction and may be aligned in line with the rotation axis RA. Also, the cavity 1101 of the handle portion 110 and the cavity 1301 of the rotating body 130 may have matching cross-sectional shapes facing each other. Through these cavities 1101 and 1301, the suction tube 140 may extend from the inside of the rotating body 130 to the inside of the handle portion 110.

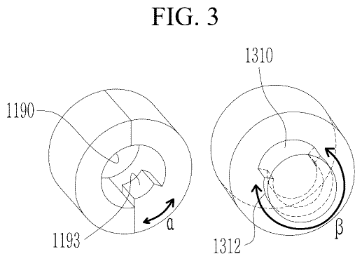

FIG. 3 is a view illustrating the protrusion and recess for limiting the rotation range of the male-female coupling structure shown in FIG. 2. FIG. 3 is a schematic view of the female part 1190 of the handle portion 110 and the male part 1310 of the rotating body 130, omitting the surrounding structure for convenience of illustration.

Referring to FIG. 3, the protrusion 1193 is formed on the inner circumferential surface of the female part 1190, and the recess 1312 may be formed on the outer circumferential surface of the male part 1310. Here, based on the central axis, the first central angle of the protrusion 1193 is smaller than the second central angle of the recess 1312. Therefore, the surface length (arc length) (α) of the protrusion 1193 along the rotation direction is formed to be smaller than the surface length (arc length) (β) of the recess 1312 along the rotation direction.

When the female part 1190 of the handle portion 110 and the male part 1310 of the rotating body 130 are coupled, the protrusion 1193 is accommodated in the recess 1312. At this time, the surface of the protrusion 1193 is positioned in contact with the surface of the recess 1312. When the rotating body 130 rotates around the rotation axis RA, the protrusion 1193 can move within the recess 1312. However, the recess 1312 has steps formed on both sides in the rotation direction that act as stoppers, which can limit the movement of the protrusion 1193.

Therefore, the surface length (α) of the protrusion 1193 and the surface length (β) of the recess 1312 can be considered to set the rotation range of the rotating body 130. The target rotation range of the rotating body 130 can be calculated as the difference (β−α) between the surface length (β) of the recess 1312 and the surface length (α) of the protrusion 1193.

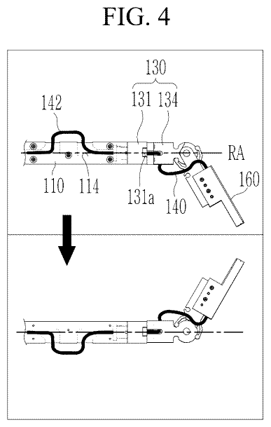

FIG. 4 is a view illustrating the operation of the handheld tissue suturing device shown in FIG. 1. From the state in the upper diagram of FIG. 4, if the rotating body 130 of the handheld tissue suturing device 100 is rotated 180 degrees around the rotation axis, it may become the state in the lower diagram.

Referring to FIG. 4, in the handheld tissue suturing device 100, the rotating body 130 can be rotated around the rotation axis RA with respect to the handle portion 110. The rotating body 130 includes a first body part 131 adjacent to the handle portion 110 and a second body part 134 adjacent to the vacuum tweezer 160. When the rotating body 130 rotates, the first body part 131 and the second body part 134 may rotate together as a unit.

In the handheld tissue suturing device 100, the suction tube 140 is connected from the vacuum tweezer 160 through the rotating body 130 to the handle portion 110. The suction tube 140 is arranged to pass through the through hole 131a of the first body part 131 when passing through the rotating body 130. The through hole 131a may be formed along the rotation axis RA. Therefore, the suction tube 140 passing through the through hole 131a may extend coaxially with the rotation axis RA. The suction tube 140 extending to the handle portion 110 may be connected to a suction pump (not shown) or the like to perform an air suction function.

As the suction tube 140 passing through the rotating body 130 is coaxial with the rotation axis RA, the suction tube 140 may not become twisted or kinked even if the rotating body 130 rotates around the rotation axis RA. When the rotating body 130 rotates, the vacuum tweezer 160 fixed to the second body part 134 of the rotating body 130 may also rotate together. Therefore, the vacuum tweezer 160 pointing downward to the right may be positioned to point upward to the right after the rotation of the rotating body 130.

Also, the handle portion 110 may have at least a pair of tube receiving grooves 114 that accommodate the suction tube 140 and are blocked from each other inside. The suction tube 140 may be arranged so that the portion connecting between the pair of tube receiving grooves 114 passes along the outer surface of the handle portion 110. Therefore, the suction tube 140 may extend into the tube receiving groove 114 of the handle portion 110 and then have a portion exposed to pass along the outer surface of the handle portion 110. This exposed portion of the suction tube 140 can stop air suction when pressed by the user and can function as a tube switch 142.

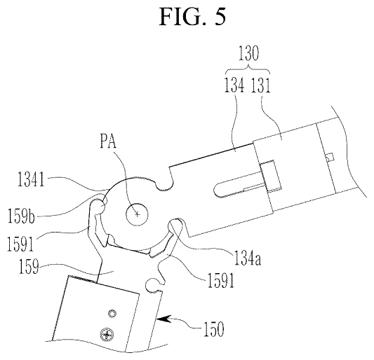

FIG. 5 is a partial side view illustrating the position fixing structure of the rotating body and the finger part of the handheld tissue suturing device shown in FIG. 1.

Referring to FIG. 5, the second body part 134 of the rotating body 130 and the pivot end 159 of the finger part 150 may have a position fixing structure that limits the driving range in the pivot direction and fixes the position. The position fixing structure may include an inner diameter part 1341 positioned around the pivot axis PA and an outer diameter part 1591 arranged to surround a portion of the outer circumference of the inner diameter part 1341. The inner diameter part 1341 may have grooves 134a on its outer circumference, and the outer diameter part 1591 may have protrusions 159b corresponding to the grooves 134a.

The outer diameter part 1591 may include a pair of arms extending in opposite directions along the outer circumference of the inner diameter part 1341. The end of each of the pair of arms may include a protrusion 159b protruding toward the inner diameter part 1341. For example, the outer diameter part 1591 may have one protrusion 159b at the end of each of the pair of arms, and the inner diameter part 1341 may have four grooves 134a on its outer circumference. Therefore, the outer diameter part 1591 may be fixed in position as two protrusions 159b are fixed in two grooves 134a while pivoting. If two protrusions 159b are fixed in two other grooves 134a as the outer diameter part 1591 pivots, it may be fixed in another position. The pair of arms may have elasticity to be variable in the radial direction of the inner diameter part 1341.

The second body part 134 of the rotating body 130 may include the inner diameter part 1341, and the finger part 150 may include the outer diameter part 1591. Therefore, even if the protrusions 159b are inserted into the grooves 134a, if external force is applied, the pair of arms of the outer diameter part 1591 may spread apart, releasing the fixed state, allowing the finger part 150 to pivot.

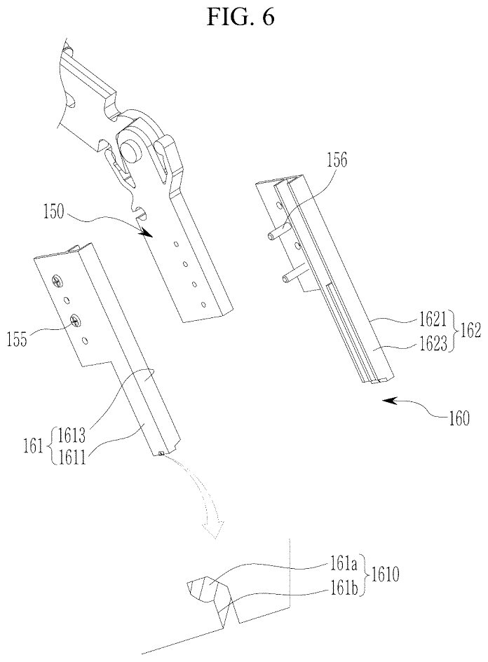

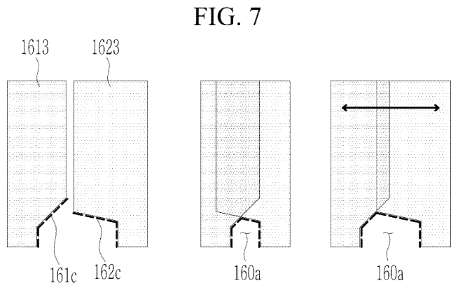

FIG. 6 is a partial exploded perspective view illustrating the finger part and the vacuum tweezer of the handheld tissue suturing device shown in FIG. 1. FIG. 7 is a view illustrating the configuration of the vacuum tweezer of the handheld tissue suturing device shown in FIG. 1.

Referring to FIG. 6, the vacuum tweezer 160 may be coupled to the finger part 150 of the handheld tissue suturing device 100. The vacuum tweezer 160 may be formed by the coupling of a first piece part 161 and a second piece part 162 on both sides of the finger part 150. Coupling pins 155 and 156 are arranged between the first piece part 161 and the second piece part 162, and the coupling pins 155 and 156 may be arranged to penetrate the finger part 150 transversely. For example, four coupling pins 155 and 156 may connect between the first piece part 161 and the second piece part 162. Two coupling pins 155 have their heads positioned on the first piece part 161 and extend to the second piece part 162, and the remaining two coupling pins 156 have their heads positioned on the second piece part 162 and may extend to the first piece part 161.

The first piece part 161 of the vacuum tweezer 160 may include a first coupling plate 1611 and a first contour forming plate 1613. The second piece part 162 of the vacuum tweezer 160 may include a second coupling plate 1621 and a second contour forming plate 1623. The first coupling plate 1611 and the second coupling plate 1621 may be attached adjacent to both sides of the finger part 150. The first contour forming plate 1613 extends from the first coupling plate 1611 in a direction perpendicular to the first coupling plate 1611, and the second contour forming plate 1623 may extend from the second coupling plate 1621 in a direction perpendicular to the second coupling plate 1621. The first contour forming plate 1613 and the second contour forming plate 1623 each may be formed in multiple pieces. The first contour forming plate 1613 and the second contour forming plate 1623 may extend to face each other and be arranged so that their respective opposing ends partially overlap. Multiple first contour forming plates 1613 and multiple second contour forming plates 1623 may partially overlap to form a conduit extending in the longitudinal direction. The suction tube 140 may be connected to the top of this conduit.

A needle hole 1610 may be located at the longitudinal lower end of each of the first coupling plate 1611 and the second coupling plate 1621. The needle hole 1610 may be formed to penetrate the first coupling plate 1611 or the second coupling plate 1621 in a direction perpendicular to the first coupling plate 1611 or the second coupling plate 1621. The needle hole 1610 has a first opening section 161a and a second opening section 161b, and the second opening section 161b is open at the lower edge of the first coupling plate 1611 or the second coupling plate 1621.

The first opening section 161a of the needle hole 1610 is an opening through which a needle used for tissue suturing passes, and the second opening section 161b of the needle hole 1610 is an opening for removing the suture thread connected to the needle. Here, the width of the connecting portion of the first opening section 161a and the second opening section 161b is smaller than the maximum width of the first opening section 161a. This can prevent a needle passing through the first opening section 161a from deviating from the needle hole 1610 before completely passing through the first coupling plate 1611 and the second coupling plate 1621.

Referring to FIG. 7, the first contour forming plate 1613 has a first concave part 161c that is concavely cut out at the lower right, and the second contour forming plate 1623 may have a second concave part 162c that is concavely cut out at the lower left. Here, the first concave part 161c and the second concave part 162c may have asymmetric contour parts. The first concave part 161c and the second concave part 162c may be integrated to form a single suturing area 160a when the first piece part 161 and the second piece part 162 are coupled. In the suturing area 160a, the edge farthest in the longitudinal direction from the lower edge of the vacuum tweezer 160 may be positioned offset to one side with respect to the width-wise center of the suturing area 160a.

When multiple first contour forming plates 1613 and multiple second contour forming plates 1623 overlap, the suturing area 160a formed by the first concave part 161c and the second concave part 162c may be formed as a space. Separated tissues that are the target of suturing may be positioned in the suturing area 160a. The size of the suturing area 160a may vary depending on the degree of overlap between the first contour forming plate 1613 and the second contour forming plate 1623.

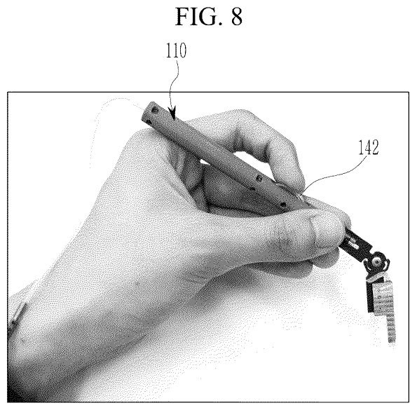

FIG. 8 is an image photograph showing the state of holding the handheld tissue suturing device shown in FIG. 1 by hand.

Referring to FIG. 8, the handheld tissue suturing device 100 according to this embodiment may be used by gripping the handle portion 110 with one hand. At this time, the air suction can be controlled by pressing the tube switch 142 exposed on the outer surface of the handle portion 110 with the index finger.

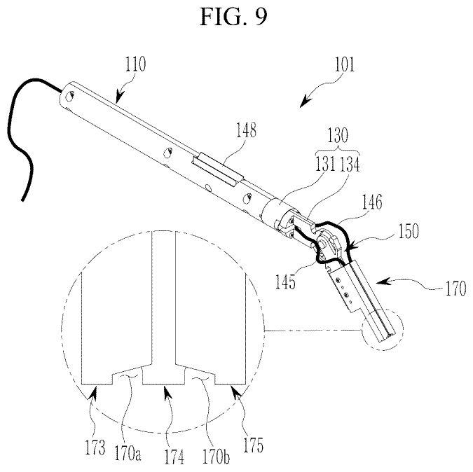

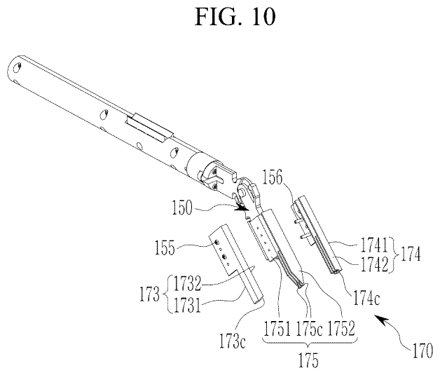

FIG. 9 is a perspective view illustrating a handheld tissue suturing device according to another embodiment. FIG. 10 is a partial exploded perspective view illustrating the finger part and the vacuum tweezer of the handheld tissue suturing device shown in FIG. 9.

Referring to FIG. 9, the handheld tissue suturing device 101 according to this embodiment includes, like the handheld tissue suturing device 100 shown in FIG. 1, a handle portion 110, a rotating body 130, and a finger part 150. A vacuum tweezer 170 used for tissue suturing is fixed to the finger part 150, and suction tubes 145 and 146 extending from the vacuum tweezer 170 to the handle portion 110 may be connected to the vacuum tweezer 170.

Referring to FIG. 10, in this embodiment, the vacuum tweezer 170 may be formed by the coupling of a third piece part 173, a fourth piece part 174, and a fifth piece part 175 on both sides of the finger part 150. The fifth piece part 175 may be positioned between the third piece part 173 and the fourth piece part 174. Coupling pins 155 and 156 are arranged between the third piece part 173 and the fourth piece part 174, and the coupling pins 155 and 156 may be arranged to penetrate the finger part 150 and the fifth piece part 175 transversely. For example, four coupling pins 155 and 156 may connect between the third piece part 173 and the fourth piece part 174. Two coupling pins 155 have their heads positioned on the third piece part 173 and extend to the fourth piece part 174, and the remaining two coupling pins 156 have their heads positioned on the fourth piece part 174 and may extend to the third piece part 173.

The third piece part 173 of the vacuum tweezer 170 may include a third coupling plate 1731 and a third contour forming plate 1732. The fourth piece part 174 of the vacuum tweezer 170 may include a fourth coupling plate 1741 and a fourth contour forming plate 1742. The fifth piece part 175 of the vacuum tweezer 170 may include a fifth coupling plate 1751 and a fifth contour forming plate 1752.

The fifth coupling plate 1751 may be attached adjacent to one side of the finger part 150. The third coupling plate 1731 may be attached adjacent to one side of the fifth coupling plate 1751, and the fourth coupling plate 1741 may be attached adjacent to the other side of the finger part 150. The third contour forming plate 1732 may extend from the third coupling plate 1731 in a direction perpendicular to the third coupling plate 1731, and the fourth contour forming plate 1742 may extend from the fourth coupling plate 1741 in a direction perpendicular to the fourth coupling plate 1741. The fifth contour forming plate 1752 may extend from the fifth coupling plate 1751 in a direction perpendicular to it.

Each of the third contour forming plate 1732 and the fourth contour forming plate 1742 may be formed in multiple pieces. The fifth contour forming plate 1752 may also be formed in multiple pieces. The third contour forming plate 1732 and the fourth contour forming plate 1742 may extend in directions facing each other. The fifth contour forming plate 1752 may extend in directions facing each of the third contour forming plate 1732 and the fourth contour forming plate 1742, thus extending to both sides. Therefore, both ends of the fifth contour forming plate 1752 may be arranged to partially overlap with the end of the third contour forming plate 1732 and the end of the fourth contour forming plate 1742, respectively. Multiple third contour forming plates 1732 and multiple fifth contour forming plates 1752 may partially overlap to form a first conduit extending in the longitudinal direction, and multiple fourth contour forming plates 1742 and multiple fifth contour forming plates 1752 may partially overlap to form a second conduit extending in the longitudinal direction. The first suction tube 145 and the second suction tube 146 may each be connected to the top of this first conduit and second conduit.

The third contour forming plate 1732 has a third concave part 173c that is concavely cut out at the lower right, and the fourth contour forming plate 1742 may have a fourth concave part 174c that is concavely cut out at the lower left. The fifth contour forming plate 1752 may have a fifth concave part 175c that is concavely cut out at the lower left and right. Here, the third concave part 173c and the fourth concave part 174c may have symmetrical contour parts with each other. The fifth concave part 175c may have asymmetrical contour parts with each of the third concave part 173c and the fourth concave part 174c.

The third concave part 173c and the fifth concave part 175c may be integrated to form a single first suturing area 170a when the third piece part 173 and the fifth piece part 175 are coupled. The fourth concave part 174c and the fifth concave part 175c may be integrated to form a single second suturing area 170b when the fourth piece part 174 and the fifth piece part 175 are coupled. In the first and second suturing areas 170a and 170b, the edge farthest in the longitudinal direction from the lower edge of the vacuum tweezer 170 may be positioned offset to one side with respect to the width-wise center of the first and second suturing areas 170a and 170b.

When multiple third contour forming plates 1732 and multiple fifth contour forming plates 1752 overlap, the first suturing area 170a formed by the third concave part 173c and the fifth concave part 175c may be formed as a space. When multiple fourth contour forming plates 1742 and multiple fifth contour forming plates 1752 overlap, the second suturing area 170b formed by the fourth concave part 174c and the fifth concave part 175c may be formed as a space. Separated tissues that are the target of suturing may be positioned in the first suturing area 170a and the second suturing area 170b. The size of the first suturing area 170a may vary depending on the degree of overlap between the third contour forming plate 1732 and the fifth contour forming plate 1752, and the size of the second suturing area 170b may vary depending on the degree of overlap between the fourth contour forming plate 1742 and the fifth contour forming plate 1752.

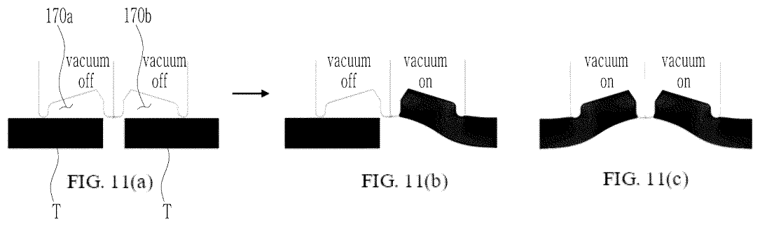

FIG. 11(a) to 11(c) provide views illustrating the configuration of the vacuum tweezer of the handheld tissue suturing device shown in FIG. 9.

The first suction tube 145 may be connected to the first conduit formed by the partial overlap of multiple third contour forming plates 1732 and multiple fifth contour forming plates 1752. The second suction tube 146 may be connected to the second conduit formed by the partial overlap of multiple fourth contour forming plates 1742 and multiple fifth contour forming plates 1752. The first suction tube 145 and the second suction tube 146 may be controlled separately from each other. Therefore, if the first suction tube 145 is closed and the second suction tube 146 is open, only the suturing target T positioned in the second suturing area 170b is sucked (see FIG. 11(b)), and if the first suction tube 145 is open and the second suction tube 146 is closed, only the suturing target T positioned in the first suturing area 170a may be sucked. If both the first suction tube 145 and the second suction tube 146 are open, the suturing target C positioned in the first suturing area 170a and the suturing target C positioned in the second suturing area 170b may be sucked together (see FIG. 11(c)).

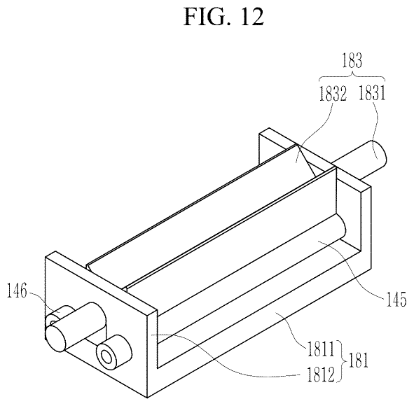

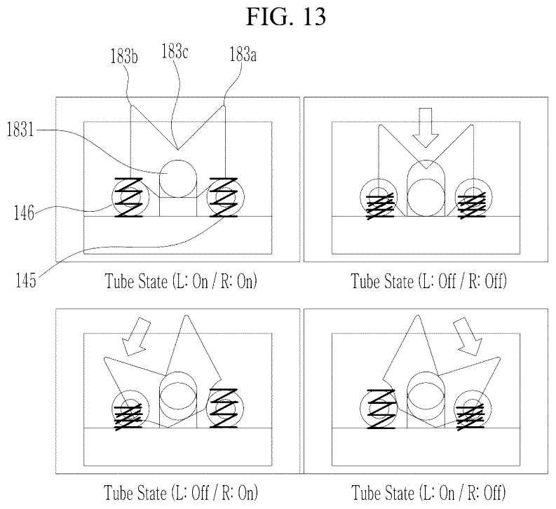

FIG. 12 is a perspective view illustrating the multiple tube control switch of the handheld tissue suturing device shown in FIG. 9. FIG. 13 is a schematic view illustrating the operation of the multiple tube control switch shown in FIG. 12.

The handheld tissue suturing device 101 according to this embodiment has a first suction tube 145 and a second suction tube 146, and these suction tubes may each be individually controlled. For this purpose, the handheld tissue suturing device 101 may be equipped with a multiple tube control switch 180 on the handle portion 110.

Referring to FIG. 12, the multiple tube control switch 180 may include a tube fixing frame 181 and a pressing member 183. The tube fixing frame 181 includes a bottom plate 1811 and a side plate 1812, and the pressing member 183 may include a guide protrusion 1831 and a pressing button 1832. The guide protrusion 1831 of the pressing member 183 may be coupled to the tube fixing frame 181 to be movable up and down along a slot formed in the side plate 1812. The first suction tube 145 and the second suction tube 146 may be positioned between the bottom plate 1811 of the tube fixing frame 181 and the pressing button 1832. The first suction tube 145 and the second suction tube 146 are flexible and may have elasticity. Therefore, the pressing button 1832 may be maintained in a state lifted upward by the first suction tube 145 and the second suction tube 146 when there is no external force. When external force is applied to the pressing button 1832, it may be pressed down to close the first suction tube 145 or the second suction tube 146.

Referring to FIG. 13, the pressing button 1832 may include a first peak 183a and a second peak 183b that protrude separately upward. A concavely depressed valley 183c may be formed between the first peak 183a and the second peak 183b. The first suction tube 145 and the second suction tube 146 may be arranged under the first peak 183a and the second peak 183b, respectively. Therefore, when the pressing button 1832 is not pressed, the first suction tube 145 and the second suction tube 146 may each be in an open state (see upper left). When pressing the valley 183c of the pressing button 1832, the entire first suction tube 145 and second suction tube 146 may be pressed to be in a closed state (see upper right). When pressing only the first peak 183a of the pressing button 1832 and not pressing the second peak 183b, the first suction tube 145 may be closed and the second suction tube 146 may be in an open state (see lower left). When pressing only the second peak 183b of the pressing button 1832 and not pressing the first peak 183a, the second suction tube 146 may be closed and the first suction tube 145 may be in an open state (see lower right).

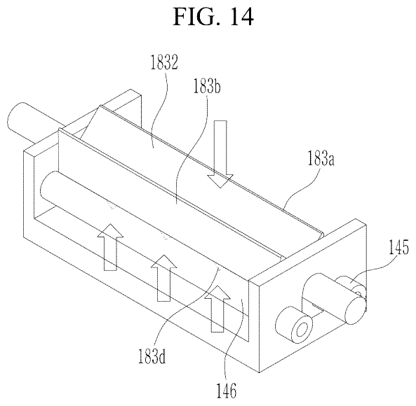

FIG. 14 is a perspective view illustrating a modification of the multiple tube control switch shown in FIG. 12.

Referring to FIG. 14, the multiple tube control switch 180′ according to this modification may include multiple protrusions 183d protruding from the lower surface of the pressing button 1832. The multiple protrusions 183d may be arranged at portions where the pressing button 1832 contacts the first suction tube 145 and the second suction tube 146, respectively. Therefore, the multiple protrusions 183d may be arranged to correspond to the vertical downward of each of the first peak 183a and the second peak 183b. Each of the multiple protrusions 183d may have a pointed end shape. This allows the first suction tube 145 and the second suction tube 146 to maintain their position without shaking left and right when placed under the pressing button 1832.

Although the preferred embodiments of the present disclosure have been described above, the present disclosure is not limited thereto, and it is possible to practice it in various modifications within the scope of the claims, the description of the disclosure, and the accompanying drawings, which also belongs to the scope of the present disclosure.

### DESCRIPTION OF SYMBOLS

100, 101: tissue suturing device
110: handle portion
114: tube receiving groove
119: connection end
130: rotating body
131: first body part
134: second body part
140: suction tube
142: tube switch
145: first suction tube
146: second suction tube
150: finger part
155, 156: coupling pins
159: pivot end
160: vacuum tweezer
161: first piece part
162: second piece part
170: vacuum tweezer
173: third piece part
174: fourth piece part
175: fifth piece part
180, 180′: tube control switch
181: tube fixing frame
183: pressing member

What is claimed is:

1. A handheld tissue suturing device, comprising:
a handle portion extending in a first direction;
a rotating body including a first body part and a second body part positioned on opposite sides of each other, wherein the first body part is connected to a connection end of the handle portion, and configured to be rotatable around a rotation axis extending in the first direction;
a finger part including a pivot end connected to the second body part of the rotating body and configured to be pivotable around a pivot axis extending in a second direction intersecting the rotation axis;
a vacuum tweezer fixed to the finger part; and
a suction tube extending from the vacuum tweezer through the rotating body to the handle portion and forming an air suction path.

2. The handheld tissue suturing device of claim 1, wherein
the suction tube is configured to extend through at least a portion of the rotating body along the rotation axis.

3. The handheld tissue suturing device of claim 1, wherein
the connection end of the handle portion and the first body part of the rotating body have a male-female coupling structure capable of relative rotation around the rotation axis.

4. The handheld tissue suturing device of claim 3, wherein
the connection end of the handle portion includes a female part, and the first body part of the rotating body includes a male part.

5. The handheld tissue suturing device of claim 3, wherein
the male-female coupling structure has a rotation limiting structure configured to limit a rotation range according to a rotation direction.

6. The handheld tissue suturing device of claim 5, wherein
the rotation limiting structure includes a protrusion having a first central angle in the rotation direction, and a recess having a second central angle larger than the first central angle, wherein the protrusion is accommodated in the recess to move in the rotation direction.

7. The handheld tissue suturing device of claim 1, wherein
the handle portion and the rotating body each have a cavity inside, and
the cavities are configured to have a coaxial central axis and to communicate with each other.

8. The handheld tissue suturing device of claim 7, wherein
the cavities are configured to be connected at adjacent portions of the handle portion and the rotating body.

9. The handheld tissue suturing device of claim 7, wherein
the coaxial central axis of the cavities extends in the first direction and is aligned in line with the rotation axis.

10. The handheld tissue suturing device of claim 1, wherein
the second body part of the rotating body and the pivot end of the finger part have a position fixing structure that limits a driving range in a pivot direction and fixes a position of the second body part of the rotating body and the pivot end of the finger part.

11. The handheld tissue suturing device of claim 10, wherein
the position fixing structure includes an inner diameter part positioned around the pivot axis and having grooves on an outer circumference of the inner diameter part, and an outer diameter part arranged to surround a portion of the outer circumference of the inner diameter part and having protrusions corresponding to the grooves.

12. The handheld tissue suturing device of claim 11, wherein
the outer diameter part includes a pair of arms extending in opposite directions along the outer circumference of the inner diameter part, and the end of each of the pair of arms includes a protrusion protruding toward the inner diameter part.

13. The handheld tissue suturing device of claim 12, wherein
the pair of arms have elasticity to move in a radial direction of the inner diameter part.

14. The handheld tissue suturing device of claim 1, wherein
the vacuum tweezer includes a first piece part and a second piece part each coupled on opposite sides of the finger part.

15. The handheld tissue suturing device of claim 14, wherein
the first piece part includes a first coupling plate attached adjacent to one side of the finger part and a first contour forming plate bent and extended from the first coupling plate,
the second piece part includes a second coupling plate attached adjacent to the other side of the finger part and a second contour forming plate bent and extended from the second coupling plate, and
the first contour forming plate and the second contour forming plate extend to face each other and are arranged so that their respective opposing ends overlap each other.

16. The handheld tissue suturing device of claim 15, wherein
a needle hole penetrating the first coupling plate and the second coupling plate is located at a longitudinal lower end of each of the first coupling plate and the second coupling plate,
the needle hole has a first opening section and a second opening section connected to the first opening section and open from a lower edge of the first coupling plate or the second coupling plate, and
a width of a connecting portion of the first opening section and the second opening section is smaller than a maximum width of the first opening section.

17. The handheld tissue suturing device of claim 15, wherein
the first contour forming plate and the second contour forming plate have a first concave part and a second concave part with asymmetric contour shapes at their lower parts adjacent to edges facing each other, and
the first concave part and the second concave part are integrated to form a suturing area when the first piece part and the second piece part are coupled.

18. The handheld tissue suturing device of claim 1, wherein
the handle portion has at least a pair of tube receiving grooves that accommodate the suction tube and are blocked from each other inside, and
the handle portion includes a tube switch where a portion of the suction tube connecting between the at least pair of tube receiving grooves is arranged to pass along an outer surface of the handle portion.

19. The handheld tissue suturing device of claim 1, wherein
the suction tube includes a first suction tube and a second suction tube forming different suction paths, and
the handle portion includes a multiple tube control switch configured to individually open or close the first suction tube and the second suction tube.

20. The handheld tissue suturing device of claim 19, wherein
the multiple tube control switch includes a tube fixing frame supporting the first suction tube and the second suction tube, and a pressing member arranged to face the tube fixing frame with the first suction tube and the second suction tube interposed.
